(19) United States
(12) Patent Application Publication
(10) Pub. No.: US 2001/0031751 A1
(43) Pub. Date: Oct. 18, 2001

(54) Pharmaceuticals

(75) Inventors: Brian David Bush, Haverford, PA (US); Michael Fedouloff, London (GB); Frank Hossner, Stevenage (GB)

Correspondence Address: GLAXOSMITHKLINE, Corporate Intellectual Property - UW2220, P.O. Box 1539, King of Prussia, PA, 19406-0939, US

(73) Assignee: SmithKline Beecham p.l.c.

(21) Appl. No.: 09/845,884

(22) Filed: Apr. 30, 2001

Related U.S. Application Data
(63) Continuation of application No. 09/555,851, filed on Jul. 13, 2000, now abandoned.
Application No. 09/555,851 is a 371 of international application No. PCT/EP98/07764, filed on Dec. 1, 1998.

(30) Foreign Application Priority Data
Dec. 5, 1997 (GB) 9725933.7

(51) Int. Cl. A61K 31/5365; C07D 498/06

(52) U.S. Cl. 514/230.2; 544/89

(57) ABSTRACT

Novel oxazino[3,2-a]indole-10-carboxamide derivatives having 5-HT₄ receptor antagonist activity and useful in the treatment of gastrointenstinal disorders, cardiovascular disorders and CNS disorders, inclusing irritable bowel syndrome.

This invention relates to novel compounds having pharmacological activity, to a process for their preparation and to their use as pharmaceuticals.

EP-A429984 (Nisshin Flour Milling Co., Ltd.) describes indole derivatives having 5-HT₃ receptor antagonist activity.

European Journal of Pharmacology 146 (1988), 187-188, and Naunyn-Schmiedeberg's Arch. Pharmacol. (1989) 340:403-410, describe a non classical 5-hydroxytryptamine receptor, now designated the 5-HT₄ receptor, and that ICS 205-930, which is also a 5-HT₃ receptor antagonist, acts as an antagonist at this receptor.

WO 91/16045 (SmithKline and French Laboratories Limited) describes the use of cardiac 5-HT₄ receptor antagonists in the treatment of atrial arrhythmias and stroke.

EP-A-501322 (Glaxo Group Limited) describes indole derivatives having 5-HT₄ antagonist activity.

W093/18036 (SmithKline Beecham plc) describes certain condensed indole derivatives having 5-HT₄ receptor antagonist activity. Among these derivatives the compound N-[(1-ⁿbutyl-4-piperidyl)methyl]-3,4-dihydro-2H-[1,3]oxazino[3,2-a]indole-10-carboxamide has been found to have potent 5-HT⁴ receptor antagonist activity and to be of therapeutic value in the treatment of irritable bowel syndrome. In the evaluation of this compound we have found that one of its metabolites, namely the compound of formula (I) below, itself has potent 5-HT₄ receptor antagonist activity.

Accordingly, the present invention provides (±)-N-[(1-butyl-4-piperidinyl)methyl]-3,4-dihydro-4-hydroxy-2H-[1,3 ]oxazino[3,2-a]indole-10-carboxamide i.e. the compound of formula (I), or a pharmaceutically acceptable salt thereof:

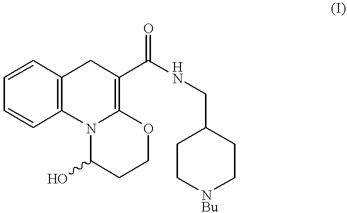

The pharmaceutically acceptable salts of the compound of the formula (I) include acid addition salts with conventional acids such as hydrochloric, hydrobromic, boric, phosphoric. sulphuric acids and pharmaceutically acceptable organic acids such as acetic, tartaric, maleic, citric, succinic, benzoic, ascorbic, methanesulphonic, α-keto glutaric, α-glycerophosphoric, and glucose-1-phosphoric acids.

Examples of pharmaceutically acceptable salts include quaternary derivatives of the compound of formula (I) such as the compounds quaternised by compounds R^{x}-T wherein R^{X} is C¹⁻⁶ alkyl, phenyl-C¹⁻⁶ alkyl or C₅₋₇ cycloalkyl, and T is a radical corresponding to an anion of an acid. Suitable examples of R^{x} include methyl, ethyl and n- and iso-propyl; and benzyl and phenethyl. Suitable examples of T include halide such as chloride, bromide and iodide.

Examples of pharmaceutically acceptable salts also include internal salts such as N-oxides.

The compound of the formula (I) and its pharmaceutically acceptable salts, (including quaternary derivatives and N-oxides) may also form pharmaceutically acceptable solvates, such as hydrates, which are included wherever the compound of formula (I) or a salt thereof is herein referred to. The compound of formula (I) is a racemate. The present invention also covers the corresponding individual (+) and (−) enantiomers.

The compound of formula (I) may be prepared as described in the Example below or by other conventional coupling of the indole moiety with Z, for example by reacting 1-n-butyl-4-piperidinyl methylamine with (+/−)-methyl 3,4-dihydro-4-(protected)hydroxy-2H-[1,3]oxazino [3,2-a]indole-10-carboxylate; the 4-hydroxy group is advantageuosly protected by an appropriate silyl group. Suitable methods are as described in GB 2125398A (Sandoz Limited), GB 1593146A and EP-A-36269 (Beecham Group p.l.c.), EP-A-429984 (Nisshin Flour Milling Co.) and EP-A-328200 (Merck Sharp & Dohme Limited). Reference is also made to EP-A-501322 (Glaxo Group Limited). It will be appreciated that the formation of O-containing ring may be carried out before or after coupling.

Aza(bi)cyclic side chain intermediates are known compounds or may be prepared according to the methods described in PCT/GB92/01519 and /01612 (SmithKline Beecham p.l.c.).

The compounds of the present invention are 5-HT₄ receptor antagonists and it is thus believed may generally be used in the treatment or prophylaxis of gastrointestinal disorders, cardiovascular disorders and CNS disorders.

They are of potential interest in the treatment of irritable bowel syndrome (IMS), in particular the diarrhoea aspects of IBS, i.e., these compounds block the ability of 5-HT to stimulate gut motility via activation of enteric neurones. In animal models of IBS, this can be conveniently measured as a reduction of the rate of defaecation. They are also of potential use in the treatment of urinary incontinence which is often associated with IBS.

They may also be of potential use in other gastrointestinal disorders, such as those associated with upper gut motility, and as antiemetics. In particular, they are of potential use in the treatment of the nausea and gastric symptoms of gastro-oesophageal reflux disease and dyspepsia. Antiemetic activity is determined in known animal models of cytotoxic-agent/radiation induced emesis.

Specific cardiac 5-HT₄ receptor antagonists which prevent atrial fibrillation and other atrial arrhythmias associated with 5-HT, would also be expected to reduce occurrence of stroke (see A. J. Kaumann 1990, Naumyn-Schmiedeberg's Arch. Pharmacol. 342, 619-622, for appropriate animal test method).

It is believed that platelet-derived 5-HT induces atrial arrhythmias which encourage atrial fibrillation and atrial disorders are associated with symptomatic cerebral and sytemic embolism. Cerebral embolism is the most common cause of ischaemic stroke and the heart the most common source of embolic material. Of particular concern is the frequency of embolism associated with atrial fibrillation.

Anxiolytic activity is likely to be effected via the hippocampus (Dumuis et al 1988, Mol Pharmacol., 34, 880-887). Activity may be demonstrated in standard animal models, the social interaction test and the X-maze test.

Migraine sufferers often undergo situations of anxiety and emotional stress that precede the appearance of headache (Sachs, 1985, Migraine, Pan Books. London). It has also been observed that during and within 48 hours of a migraine attack, cyclic AMP levels are considerably increased in the cerebrospinal fluid (Welch et al., 1976, Headache 16, 160-167). It is believed that a migraine, including the prodomal phase and the associated increased levels of cyclic AMP are related to stimulation of 5-HT₄ receptors, and hence that administration of a 5-HT₄ antagonist is of potential benefit in relieving a migraine attack

The invention also provides a pharmaceutical composition comprising the compound of formula (I), or a pharmaceutically acceptable salt thereof, and a pharmaceutically acceptable carrier.

Such compositions are prepared by admixture and are usually adapted for enteral such as oral, nasal or rectal, or parenteral administration, and as such may be in the form of tablets, capsules, oral liquid preparations, powders, granules, lozenges, reconstitutable powders, nasal sprays, suppositories, injectable and infusable solutions or suspensions. Sublingual or transdermal administration is also envisaged. Orally administrable compositions are preferred, since they are more convenient for general use.

Tablets and capsules for oral administration are usually presented in a unit dose, and contain conventional excipients such as binding agents, fillers, diluents, tabletting agents, lubricants, disintegrants, colorants, flavourings, and wetting agents. The tablets may be coated according to well known methods in the art, for example with an enteric coating.

Suitable fillers for use include cellulose, mannitol, lactose and other similar agents. Suitable disintegrants include starch, polyvinylpolypyrrolidone and starch derivatives such as sodium starch glycollate. Suitable lubricants include, for example, magnesium stearate.

Suitable pharmaceutically acceptable wetting agents include sodium lauryl sulphate. Oral liquid preparations may be in the form of, for example, aqueous or oily suspensions, solutions, emulsions, syrups, or elixirs, or may be presented as a dry product for reconstitution with water or other suitable vehicle before use. Such liquid preparations may contain conventional additives such as suspending agents, for example sorbitol, syrup, methyl cellulose, gelatin, hydroxyethylcellulose, carboxymethylcellulose, aluminium stearate gel or hydrogenated edible fats, emulsifying agents, for example lecithin, sorbitan monooleate, or acacia; non-aqueous vehicles (which may include edible oils), for example, almond oil, fractionated coconut oil, oily esters such as esters of glycerine, propylene glycol, or ethyl alcohol; preservatives, for example methyl or propyl p-hydroxybenzoate or sorbic acid, and if desired conventional flavouring or coloring agents.

Oral liquid preparations are usually in the form of aqueous or oily suspensions. solutions, emulsions, syrups, or elixirs or are presented as a dry product for reconstitution with water or other suitable vehicle before use. Such liquid preparations may contain conventional additives such as suspending agents, emulsifying agents, non-aqueous vehicles (which may include edible oils), preservatives, and flavouring or coloring agents.

The oral compositions may be prepared by conventional methods of blending, filling or tabletting. Repeated blending operations may be used to distribute the active agent throughout those compositions employing large quantities of fillers. Such operations are, of course, conventional in the art.

For parenteral administration, fluid unit dose forms are prepared containing a compound of the present invention and a sterile vehicle. The compound, depending on the vehicle and the concentration, can be either suspended or dissolved. Parenteral solutions are normally prepared by dissolving the compound in a vehicle and filter sterilising before filling into a suitable vial or ampoule and sealing. Advantageously, adjuvants such as a local anaesthetic, preservatives and buffering agents are also dissolved in the vehicle. To enhance the stability, the composition can be frozen after filling into the vial and the water removed under vacuum.

Parenteral suspensions are prepared in substantially the same manner except that the compound is suspended in the vehicle instead of being dissolved and sterilised by exposure of ethylene oxide before suspending in the sterile vehicle. Advantageously, a surfactant or wetting agent is included in the composition to facilitate uniform distribution of the compound of the invention.

The invention further provides a method of treatment of irritable bowel syndrome, gastro-oesophagal reflux disease, dyspepsia, atrial arrhythmias and stroke, anxiety and/or migraine in mammals, such as humans, which comprises the administration of an effective amount of the compound of the formula (I) or a pharmaceutically acceptable salt thereof. In particular, the method comprises treatment of IBS or atrial arrhythmias and stroke.

An amount effective to treat the disorders hereinbefore described depends on the relative efficacies of the compounds of the invention, the nature and severity of the disorder being treated and the weight of the mammal. However, a unit dose for a 70 kg adult will normally contain 0.05 to 1000 mg for example 0.5 to 500 mg, of the compound of the invention. Unit doses may be administered once or more than once a day, for example. 2. 3 or 4 times a day, more usually 1 to 3 times a day, that is in the range of approximately 0.0001 to 50 mg/kg/day, more usually 0.0002 to 25 mg/kg/day.

No adverse toxicological effects are indicated within the aforementioned dosage ranges.

The invention also provides the compound of formula (I) or a pharmaceutically acceptable salt thereof for use as an active therapeutic substance, in particular for use as a 5-HT₄ receptor antagonist in the treatment of the disorders hereinbefore described.

The invention also provides the use of the compound of formula (I) in the manufacture of a medicament for use as a 5-HT₄ receptor antagonist in the treatment of the disorders hereinbefore described.

The following Example illustrates the preparation of the compound of formula (I) with refence to the reaction scheme below:

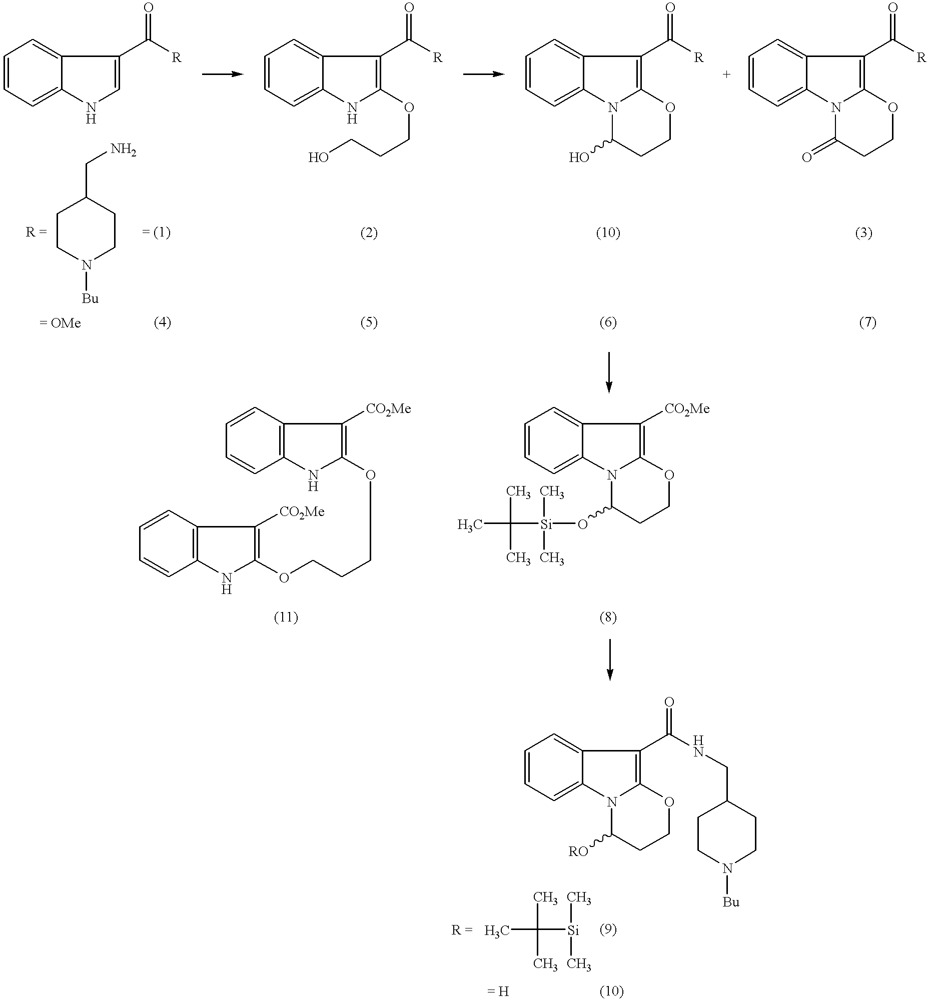

(1) N-[(1-butyl-4-piperidinyl)methyl]-1H-indole-3-carboxamide

(2) N-[(1-butyl-4-piperidinyl)methyl]-2-(3-hydroxypropoxy)-1H-indole-3-carboxamide

(3) N-[(1-butyl-4-piperidinyl)methyl]-3,4-dihydro-4-oxo-2H-[1,3]oxazino[3,2-a]indole-10-carboxamide

(4) methyl 1H-indole-3-carboxylate

(5) methyl 2-(3-hydroxypropoxy)-1H-indole-3-carboxylate

(6) methyl 3.4-dihydro-5-hydroxy-4-oxo-2H-[1,3]oxazino[3,2-α]indole-10-carboxylate

(7) methyl 3,4-dihydro-4-hydroxy-2H-[1,3]oxazino[3,2-a]indole-10-carboxylate

(8) (±)-methyl 3,4-dihydro-4-[[dimethyl-(1,1 -dimethylethyl)silyl]oxy]-2H-[1,3]oxazino[3,2-a]indole-10-carboxylate

(9) (±)-N-[(1-butyl-4-piperidinyl)methyl]-3.4-dihydro4-[[dimethyl-(1,1-dimethylethyl)silyl]oxy]-2H-[1,3]oxazino[3.2-a]indole-10-carboxamide

(10) (±)-N-[(1-butyl-4-piperidinyl)methyl]-3.4-dihydro-4-hydroxy-2H-[1,3]oxazino[3,2-a]indole-10-carboxarnide

(11) 2,2′-propane-1,3-diylbis(oxy)bis[methyl 1H-indole-3-carboxylate]

The metabolite (10) was prepared a) as a mixture and b) as a pure compound:

### Preparation a)

### Preparation of (2)

To a suspension of (1) (7.62 g, 0.024 mol) in chloroform (75 ml) at 5° C. was added N -chlorosuccinimide (3.53 g, 0.026 mol). After 30 min, 1,3-propanediol (18.5 g, 0.24 mol) was added, followed by methanesulphonic acid (0.5 g, 0.005 mol). The solution was stirred for a further 30 min, then washed with aqueous sodium carbonate solution (10% w/v, 50 ml). The chloroform layer was cooled to 0° C., the precipitate collected by filtration, obtaining 7.52 g (80%) (2).

¹H NMR (200 MHz) CD₃ OD: δ: 8.05 (m,1H), 7.25 (m,1H), 7.06 (m,2H), 4.55 (t,2H), 3.8 (t,2H), 3.0 (d,2H), 2.4 (m,2H), 1.9-2.2 (m,4H), 1.3-1.9 (m,11H),0.95 (t,3H)

### Preparation of (10) as a Mixture

To a suspension of (2) (50 mg, 0.001 mol), N-methylmorpholine-N-oxide (23 mg, 0.0015 mol), powdered 4Å molecular sieves (65 mg) in acetonitrile (2 ml) was added tetrapropylammonium perruthenate (4 mg, 0.05 mmol). The mixture was stirred for 16 hr. TLC analysis (SiO₂, MeOH) indicated metabolite (10), starting material (2), and over-oxidized diamide (3).

### Preparation b)

### Preparation of (5)

To a suspension of (4) (25.3 g, 0.144 mol) and DABCO (8.7 g, 0.078 mol) in chloroform (250 ml) at 5° C. was added N-chlorosuccinimide (21 g, 0.157 mol). After 30 min, 1,3-propanediol (110 g, 1.45 mol) was added, followed by methanesulphonic acid (3 g). The solution was allowed to warm to room temperature and stirred at room temperature for 1 hr, then washed with 10% aq. Na₂CO₃ solution, water, brine and dried (Na₂SO₄), filtered, evaporated under reduced pressure to leave an oil. Chromatography (5-50% ethyl acete/dichloromethane) gave 16.2 g (45%) (5), 3.7 g (12%) dimer (11).

¹H NMR

(5) (CD₃ OD) δ: 7.85 (m.1H). 7.25 (m.1H). 7.1 (m.2H). 4.45 (t.2H). 3.85 (m.5H), 2.1 (m.2H)

(11) (d⁶DMSO) δ: 7.8 (m,2H), 7.26 (m,2H), 7.06 (m,4H), 4.55 (t,4H), 3.68 (s,6H) (s2.3 (m,2H)

### Preparation of (6)

To a mixture of (5) (4.8 g, 0.19 mol), powdered 4Å molecular sieves (9.6 g), N-methyl morpholine-N-oxide (3.39 g, 0.029 mol) in dichloromethane/acetonitrile (10:1, 44 ml) was added tetrapropylammonium perruthenate (0.34 g, 0.00097 mol). The mixture was stirred for 18 hr, filtered through celite, and the filtrate evaporated. The residue was chromatographed (0-10% ethyl acetate/dichloromethane) obtaining 1.23 g (28%) (6), 0.52 g (11%) (7).

¹H NMR (CDCl₃)

(6) δ: 7.8 (m,1H), 7.0-7.3 (m,3H), 5.85 (br. s, 1H), 4.4-4.73 (m,2H), 3.7 (s,3H),2.1-(2.4 (m,2H)

¹H NMR (CDCl₃)

(7) δ: 8.2 ((m,1H), 7.95 (m,1H), 7.16-7.4 (m,2H), 4.66 (t,2H), 3.86 (s,3H),3.0(t,2H)

### Preparation of (8)

To a solution of (6) (2 g, 0.0081 mol) and 2,6-lutidine (4.2 ml, 0.036 mol) in dichloromethane (64 ml) at −70° C. was added TBDMS triflate (4 ml, 0.0174 mol) dropwise over 3 min. The solution was stirred for 1 hr, then allowed to warm to room temperature. The solution was re-cooled to −70° C., then methanol (9 ml) added dropwise over 5 min. The solution was diluted with dichloromethane (200 ml), washed with water, brine and dried (Na₂SO₄) to give an oil. This was chromatographed (10% ethyl acetate/dichloromethane to give 2 g (8).

¹H NMR (CDCl₃) δ: 8.1 (m,1H), 7.2-7.31 (m, 3H+CHCl₃), 6.0 (m,1H), 4.7-4.86 (m,2H), 3.95 (s,3H), 2.37-2.44 (m,1H), 2.14-2.22 (m,1H), 0.91 (s,9H), 0.29 (s,3H) 0.22 (s,3H)

### Preparation of (9)

To a solution of 1-n-butyl-4-piperidinyl methylamine (0.95 g, 0.047 mol) in toluene (2.5 ml) was added trimethylaluminium (2.3 ml, 2M in toluene, 0.046 mol) followed by (8) (1.62 g, 0.045 mol) in toluene (5 ml). The solution was heated under reflux for 4 hr, then allowed to cool to room temperature and stirred with 10% aq. NaOH solution (2 ml) for 30 min. The organic layer was washed with 10% aq. NaOH solution, water, brine, dried (Na₂SO₄), filtered and the filtrate evaporated to leave an oil. This was filtered through silica, washing with ethyl acetate, then methanol obtaining 2 g (89%) (9).

¹H NMR (CD₃OD) δ: 8.1 (m,1H), 7.3 (m,1H), 7.1 (m,2H), 6.17 (m,1H), 4.7 (m,2H), 3.0 (m,2H), 0.9-2.6 (m,29H), 0.3 (s,3H), 0.2 (s,3H)

### Preparation of (10)

A solution of (9) (0.15 g, 0.0003 mol) in water (5 ml) and glacial acetic acid (10 ml) was heated at 75° C. for 6½ hr. The solution was evaporated under reduced pressure and the residue partitioned between ethyl acetate (25 ml) and saturated aq. NaHCO3 solution (3 ml). The ethyl acetate layer was washed with further saturated aq. NaHCO₃ solution, brine, and dried (Na₂SO₄). Evaporation under reduced pressure gave (10) as a foam, 0.096 g (83%) (10).

¹H NMR (CD₃OD) δ: 8.16 (m,1H), 7.5 (m,1H), 7.2 (m,2H), 6.05 (m,1H), 4.8 (m,2H), 3.4 (m,2H), 3.1 (m,2H), 1.35-2.6 (m,15H), 1.03 (t,3H) MS (M+1) 386.3

### 5-HT₄ RECEPTOR ANTAGONIST ACTIVITY

Male Dunkin Hartley guinea-pigs, weighing 200-300 g are used. Longitudinal muscle-myenteric plexus (LMMP) preparations, 2-3cm long, are obtained from the distal colon region. These are suspended under a 0.5 g load in isolated tissue baths containing Krebs Henseleit solution (NaCl 118 mM, KCl 4.7mM KH2PO4 1.2mM MgSO4 7H2O 1.2mM, Glucose 11.1mM, NaHCO3 25mM, CaCl2 6H2O 2.5mM) bubbled with 5% CO₂ in O₂, maintained at 37° C. and containing 1uM Granisetron and 0.1uM Methiothepin to block 5HT₃ and 5HT₁-like receptors respectively. 100uM Pargyline is also added to the tissues at the start of the experiment. Tissues are left for 15 minutes to equilibrate and then exposed to 5-HT at 0.1uM every 15 minutes until a uniform response is achieved. Following a half hour period for tissues to stabilise, non-cumulative dose response curves to 5-HT are constructed in all tissues. When base-lines have returned to normal, test compounds are added in the reservoirs of the tissue set-ups and washed in to the tissues. Tissues are incubated with the antagonists for 45 minutes after which a second non-cumulative dose response curve to 5-HT is constructed.

To test for selectivity of action, compounds are tested for their ability to antagonise cholinergically-mediated contractions of the guinea-pig colon, evoked by the nicotinic receptor agonist. DMPP (1,1-dimethyl-4-phenyl-piperazinium iodide). For these experiments, tissues and equipment are set up as for the above, 5HT₄ receptor experiments. After sensitization, non-cumulative dose response curves are constructed to DMPP. Test compounds are incubated with the tissues as above, and a second dose response curve to DMPP created. Results are given as mean pKB± SEM values for each antagonists.

The compound of formula (I) was found to have a pKB value of 8.47±0.23 (n=7) and did not significantly affect DMPP-evoked contractions.

1. (±)-N-[(1-Butyl4-piperidinyl)methyl]-3,4-dihydro-4-hydroxy-2H -[1,3]oxazino[3,2-α]indole-10-carboxamide i.e. the compound of formula (I), or a pharmaceutically acceptable salt thereof:

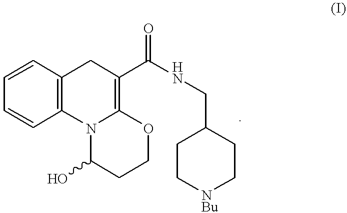

2. A process for preparing a compound of formula (I) according to claim 1 which comprises reacting 1-n-butyl-4-piperidinyl methylamine with (+/−)-methyl 3,4-dihydro-4-(protected)hydroxy-2H-[1,3]oxazino[3,2-a]indole-10-carboxylate.

3. A pharmaceutical composition comprising a compound according to claim 1, and a pharmaceutically acceptable carrier.

4. A compound according to claim 1 for use as an active therapeutic substance.

5. The use of a compound according to claim 1 in the manufacture of a medicament for use as a 5-HT₄ receptor antagonist.

6. The use according to claim 5 for use as a 5-HT₄ antagonist in the treatment or prophylaxis of gastrointestinal disorders, cardiovascular disorders and CNS disorders.

7. A method of treatment of irritable bowel syndrome, gastro-oesophagal reflux disease, dyspepsia, atrial arrhythmias and stroke, anxiety and/or migraine in mammals, such as humans, which comprises the administration of an effective amount of the compound of the formula (I) or a pharmaceutically acceptable salt thereof.
